(19) United States
(12) Patent Application Publication
(10) Pub. No.: US 2025/0235809 A1
(43) Pub. Date: Jul. 24, 2025

(54) AIR PURIFICATION SYSTEM

(71) Applicant: Jaguar Land Rover Limited, Whitley, Coventry (GB)

(72) Inventors: Waad NASSAR, Coventry, West Midlands (GB); Irina DEJANU, Coventry, West Midlands (GB)

(21) Appl. No.: 18/703,671

(22) PCT Filed: Oct. 21, 2021
(86) PCT No.: PCT/EP2021/079281

§ 371 Date: Apr. 22, 2024

(51) Int. Cl. B01D 46/44; A61L 9/22; B01D 46/00; B01D 46/56; B60H 3/00

(52) U.S. Cl. CPC B01D 46/44; A61L 9/22; B01D 46/0028; B01D 46/0032; B01D 46/0038; B01D 46/56; B60H 3/0078; A61L 2209/111; A61L 2209/14; B01D 2279/40; B01D 2279/65

(57) ABSTRACT

An air purification system for a vehicle cabin (12) of a vehicle (10) comprising a first seat arrangement (30) and a second seat arrangement (32), the air purification system comprising a first ionisation device (48) mountable in a first position (40) within the vehicle to impact air quality in a first region of the vehicle cabin (12) associated with the first seat arrangement (30); a second ionisation device (60) mountable in a second position (34) within the vehicle to impact air quality in a second region of the vehicle cabin (12) associated with the second seat arrangement (32); a user input arrangement (100; 120) configured to receive at least a first user command; and a control system (14) comprising one or more controllers, the control system configured to receive the first user command and generate a first control signal in response to the first user command to operate the air purification system in a first mode of operation in which both the first ionisation device (48) and the second ionisation device (60) are activated together.

### TECHNICAL FIELD

The present disclosure relates to an air purification system for use in a vehicle. Aspects of the invention relate to a controller, to a method, to a vehicle comprising the air purification system, and to a non-transitory, computer-readable medium.

### BACKGROUND

Air quality is becoming an increasingly important issue in the world around us. In the automotive industry, for example, customer awareness of air quality within the vehicle cabin, and the impact on health and well-being, is known to influence the customer's buying decision. Whether for business or recreation purposes, some vehicle users can spend a significant amount of time, on a daily basis, within the vehicle cabin and the requirement for a clean and healthy environment is paramount.

It is known to provide multiple air quality control features in some vehicles, including features to reduce particulate matter, harmful gases, viruses, bacteria and allergens, as well as odour and volatile organic compounds. The challenge for the vehicle manufacturer is to implement adequate air quality control features to satisfy users' awareness and concern for air quality, but to optimise systems so that they run efficiently together and with intuitive control for users. In a climate where vehicles are provided with an increasing number of technical features and accessories, ease of use by the user cannot be ignored.

It is against this background that the present invention has been devised.

### SUMMARY OF THE INVENTION

According to a first aspect of the present invention there is provided an air purification system for a vehicle cabin of a vehicle comprising a first seat arrangement and a second seat arrangement, the air purification system comprising a first ionisation device mountable in a first position within the vehicle to impact air quality in a first region of the vehicle cabin associated with the first seat arrangement; and a second ionisation device mountable in a second position within the vehicle to impact air quality in a second region of the vehicle cabin associated with the second seat arrangement. The system includes a user input arrangement configured to receive at least a first user command and a control system comprising one or more controllers, the control system configured to receive the first user command and generate a first control signal in response to the first user command to operate the air purification system in a first mode of operation in which both the first ionisation device and the second ionisation device are activated together.

The air purification system may comprise one or more controllers which collectively comprise at least one electronic processor having an electrical input for receiving the first user command; and at least one memory device electrically coupled to the at least one electronic processor and having instructions stored therein; wherein the at least one electronic processor is configured to access the at least one memory device and execute the instructions thereon so as to generate a first control signal in response to the first user command to operate the air purification system in a first mode of operation in which both the first ionisation device and the second ionisation device are activated together.

By way of example, the first position for the first ionisation device may be an instrument panel at the front of the vehicle, forward of vehicle occupants seated in the front vehicle seats. The first ionisation device may be mounted behind the instrument panel or in the vicinity of the instrument panel. The second position for the second ionisation device may be the centre console situated between the left and right side seats of the front row of vehicle seats.

Each of the first and second ionisation devices may take the form of an OH (hydroxyl) radical generator device.

The user input arrangement may be a user input device having, for example, a push button, a touch-sensitive pad/region of a touch-sensitive display screen (e.g. a personal tablet or mobile telephone), a switch, a selector, a dial or a fob, for receiving at least the first user command.

The air purification system may further comprise an air filtration system comprising a first filter and a second filter, the filtration system being operable in at least one of a recirculation mode in which air drawn into the vehicle cabin is recirculated through the main filter at least once and a fresh air mode in which air drawn into the vehicle cabin passes through the filter and the main filter only once.

The air purification system may also include a sensor system including a first sensor for sensing a concentration level of particulate matter within the vehicle cabin. The sensor system may also include a second sensor for sensing a concentration level of carbon dioxide in the vehicle cabin. Either the first or the second sensor may be provided alone or both together on the vehicle.

By way of example, in one embodiment the air purification system may comprise an air filtration system having a first filter and a second filter, the air filtration system being operable in at least one of a recirculation mode in which air drawn into the vehicle cabin is recirculated through the second filter at least once and a fresh air mode in which air drawn into the vehicle cabin passes through the second filter only once. The air purification system may comprise a sensor system which generates a sensor output in response to at least one of a concentration level of particulates within the vehicle cabin and a concentration of particulates within the external environment, the controller being configured to operate the air filtration system automatically in either the recirculation mode or the fresh air mode in response to the sensor output and the first user command.

In response to the first user command, the or each controller may be operable to activate at least one of the sensors and, in response to one or more output signals from the sensor(s), automatically select the fresh air mode or the recirculation mode.

In this way, receipt of the first user command by the controller may serve to activate both the first and second ionisation devices, and the fresh air mode or the recirculation mode of the filtration system (depending on the sensor(s) output), simultaneously.

The user input arrangement may be configured to receive a second user command, the controller being configured to receive the second user command and generate a second control signal in response to the second user command to operate the air purification system in a second mode of operation in which the first ionisation device and the second ionisation device are operated together with the air filtration system. The operation of the air filtration system may be controlled independently of the first ionisation device and the second ionisation device. The air filtration system may operate independently of the automatic control of the first ionisation device and the second ionisation device controller in response to the sensor output and/or the first user command. For example, the air filtration system may be operated in dependence on the second user command.

The filtration system may include a main filter for filtering particles having a size of up to, for example, 2.5 μm.

The air purification system may further comprise at least one carbon dioxide management device located in at least one of the first and second positions.

By way of example, the controller may be configured to generate a second signal in response to the second user command to operate the air purification system in a second mode of operation in which the first ionisation device and the second ionisation device are operated together with the air filtration system and the carbon dioxide management device. The operation of the carbon dioxide management device may be controlled independently. The carbon dioxide management device may operate independently of the automatic control of the first ionisation device and the second ionisation device controller in response to the sensor output and/or the first user command.

The user input arrangement may further include at least first and second controls for receiving respective ones of the first and second user commands to operate the air purification system in the first and the second mode of operation, respectively.

The user input arrangement may, for example, be configured to receive a third user command, the controller being configured to receive the third user command and generate a third control signal in response to the third user command to operate the air purification system in a third mode of operation in which carbon dioxide management device is operated on its own.

According to another aspect of the invention, there is provided a vehicle comprising the air purification system of the previous aspect, the vehicle comprising the vehicle cabin and the first and second seat arrangements.

For example, the first seat arrangement may be a first row of vehicle seats and the second seat arrangement may be a second row of vehicle seats behind the first row of vehicle seats. Further rows of seats may be provided behind the second row of seats. Each row of seats may include one or more seat. Therefore, the vehicle may include a third seat arrangement or further seat arrangements where the third seat arrangement may include its own ionisation device, mountable in a third position within the vehicle to impact air quality in a third zone or region of the vehicle cabin associated with the third seat arrangement.

The first ionisation device may be located behind an instrument panel forward of the first row of vehicle seats.

For example, the second ionisation device may be located in a region of the vehicle cabin between adjacent seats of the first row of vehicle seats.

The second ionisation device may be located in a centre console between adjacent seats of the first row of vehicle seats.

For example, the second ionisation device may be located behind an air vent located in the centre console.

In one embodiment, the user input arrangement may include a first user input device operable by a user seated in one of the seats of the first row and a second user input device operable by a user seated in one of the seats of the second row, and wherein both the first and second user input elements are configured to receive a first user command to operate the air purification system in the first mode of operation in which both the first ionisation device and the second ionisation device are operated together.

For example, the second user input element may be a portable user device such as a mobile telephone, a tablet or another mobile handheld device with Bluetooth® capabilities.

According to another aspect of the invention there is provided a method of controlling an air purification system for a vehicle cabin of a vehicle comprising a first seat arrangement and a second seat arrangement, a first ionisation device mountable in a first position within the vehicle to impact air quality in a first region of the vehicle cabin associated with the first seat arrangement and a second ionisation device mountable in a second position within the vehicle to impact air quality in a second region of the vehicle cabin associated with the second seat arrangement; the method comprising receiving, at a controller, a first user command which is operable to control the first ionisation device and the second ionisation device in a first mode of operation in which they are both activated together.

It will be appreciated that preferred and or optional features of the first aspect of the invention may be incorporated alone or in appropriate combination in any of the other aspects of the invention also.

According to another aspect the invention provides a non-transitory, computer-readable storage medium storing instructions thereon that, when executed by one or more electronic processors, causes the one or more electronic processors to carry out the method according to the preceding paragraph.

Any control unit or controller described herein may suitably comprise a computational device having one or more electronic processors. The system may comprise a single control unit or electronic controller or alternatively different functions of the controller may be embodied in, or hosted in, different control units or controllers. As used herein the term “controller” or “control unit” will be understood to include both a single control unit or controller and a plurality of control units or controllers collectively operating to provide any stated control functionality. To configure a controller or control unit, a suitable set of instructions may be provided which, when executed, cause said control unit or computational device to implement the control techniques specified herein. The set of instructions may suitably be embedded in said one or more electronic processors. Alternatively, the set of instructions may be provided as software saved on one or more memory associated with said controller to be executed on said computational device. The control unit or controller may be implemented in software run on one or more processors. One or more other control unit or controller may be implemented in software run on one or more processors, optionally the same one or more processors as the first controller. Other suitable arrangements may also be used.

Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. The applicant reserves the right to change any originally filed claim or file any new claim accordingly, including the right to amend any originally filed claim to depend from and/or incorporate any feature of any other claim although not originally claimed in that manner.

### BRIEF DESCRIPTION OF THE DRAWINGS

One or more embodiments of the invention will now be described, by way of example only, with reference to the accompanying drawings, in which:

FIG. 1 shows a schematic illustration of a vehicle including an air purification system according to an example of the invention;

FIG. 2 shows a schematic illustration of the control system of the air purification system FIG. 1;

FIG. 3 shows a top plan view of vehicle cabin to illustrate the seating arrangement of the vehicle in FIG. 1;

FIG. 4 is a perspective view of an instrument panel in the vehicle cabin of FIG. 3, to illustrate the position of a first ionisation device of the air purification system;

FIG. 5 is an enlarged perspective view of the ionisation device in FIG. 4;

FIG. 6 is a top view of the vehicle cabin in FIG. 3 to illustrate the position of a filtration system;

FIG. 7 is a perspective view of the first ionisation device in FIGS. 4 and 5;

FIG. 8 is a perspective view of a centre console of the vehicle cabin in FIG. 3, to illustrate the position of a second ionisation device of the air purification system;

FIG. 9 is an isolated perspective view of a part of the centre console housing in FIG. 8 to illustrate the second ionisation device;

FIG. 10 is a perspective view of an alternative arrangement to that in FIG. 9 to illustrate alternative ducting for the second ionisation device;

FIG. 11 is a perspective view from the rear of an instrument panel of the vehicle to illustrate the position of an internal sensor of the air purification system;

FIG. 12 is a perspective view from the front side of the vehicle (leaf screen) to illustrate the position of an external sensor (external to the vehicle cabin) of the air purification system;

FIG. 13 is a schematic illustration of a first user input arrangement for the air purification system; and

FIG. 14 is a schematic illustration of a second user input arrangement for the air purification system.

### DETAILED DESCRIPTION

An air purification system in accordance with an embodiment of the present invention will now be described with reference to the accompanying figures.

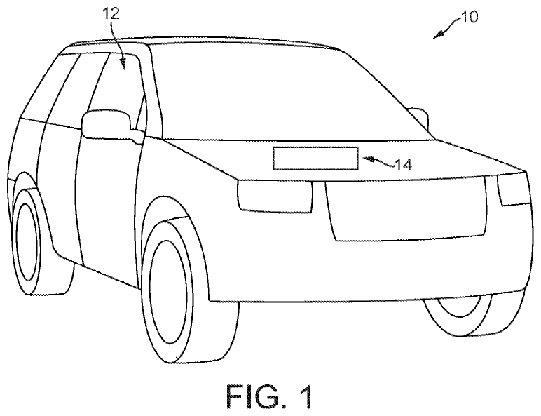

FIG. 1 shows a vehicle 10 in the form of a car. The vehicle 10 has a vehicle cabin 12. The vehicle 10 includes the air purification system for controlling the quality of air within the vehicle cabin 12. The air purification system includes several features located in various positions around the vehicle and within the vehicle cabin 12. The air purification system includes a control system, represented schematically as item 14, comprising one or more controllers. The controller may be located in any position within the vehicle and it will be appreciated that the position shown in FIG. 1 is illustrative only. The vehicle in FIG. 1 is a left-hand drive vehicle with the steering wheel 15 on the left-hand side of the vehicle cabin 12.

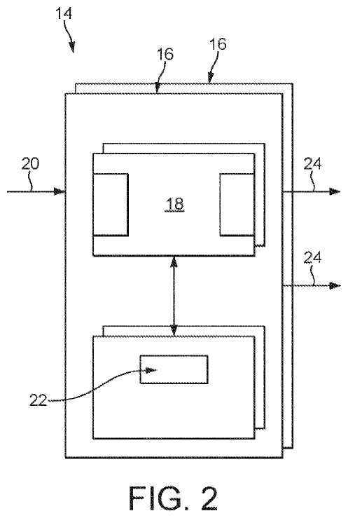

Referring to FIG. 2, the one or more controllers 16 of the control system 14 comprise at least one electronic processor 18 having an electrical input for receiving user commands 20; and at least one memory device 22 electrically coupled to the at least one electronic processor 18 and having instructions stored therein; wherein the at least one electronic processor 18 is configured to access the at least one memory device 22 and execute the instructions thereon so as to generate output control signals 24 in response to the user commands which are sent to the air purification system to operate the system in various different modes of operation, as will be described in further detail below. The input 20 of the or each controller is configured to receive data from a plurality of sources. Specifically, as will be explained in more detail later, the input 20 is configured to receive a user input command which is input by the user via a user input arrangement (not shown in FIG. 2). The user input arrangement will be described in further detail below.

It is to be understood that the or each controller 16 can comprise a control unit or computational device having one or more electronic processors (e.g., a microprocessor, a microcontroller, an application specific integrated circuit (ASIC), etc.), and may comprise a single control unit or computational device, or alternatively different functions of the or each controller may be embodied in, or hosted in, different control units or computational devices. As used herein, the term “controller,” “control unit,” or “computational device” will be understood to include a single controller, control unit, or computational device, and a plurality of controllers, control units, or computational devices collectively operating to provide the required control functionality. A set of instructions could be provided which, when executed, cause the or each controller to implement the control techniques described herein (including some or all of the functionality required for the method described herein). The set of instructions could be embedded in said one or more electronic processors of the controller(s); or alternatively, the set of instructions could be provided as software to be executed in the controller(s). A first controller or control unit may be implemented in software run on one or more processors. One or more other controllers or control units may be implemented in software run on one or more processors, optionally the same one or more processors as the first controller or control unit. Other arrangements are also useful.

The or each electronic processor 18 may comprise any suitable electronic processor (e.g., a microprocessor, a microcontroller, an ASIC, etc.) that is configured to execute electronic instructions. The or each electronic memory device 22 may comprise any suitable memory device and may store a variety of data, information, limit value(s), lookup tables or other data structures, and/or instructions therein or thereon. In an embodiment, the memory device 22 has information and instructions for software, firmware, programs, algorithms, scripts, applications, etc. stored therein or thereon that may govern all or part of the methodology described herein. The or each electronic processor 18 may access the memory device 22 and execute and/or use that or those instructions and information to carry out or perform some or all of the functionality and methodology describe herein.

The at least one memory device 22 may comprise a computer-readable storage medium (e.g. a non-transitory or non-transient storage medium) that may comprise any mechanism for storing information in a form readable by a machine or electronic processors/computational devices, including, without limitation: a magnetic storage medium (e.g. floppy diskette); optical storage medium (e.g. CD-ROM); magneto optical storage medium; read only memory (ROM); random access memory (RAM); erasable programmable memory (e.g. EPROM and EEPROM); flash memory; or electrical or other types of medium for storing such information/instructions.

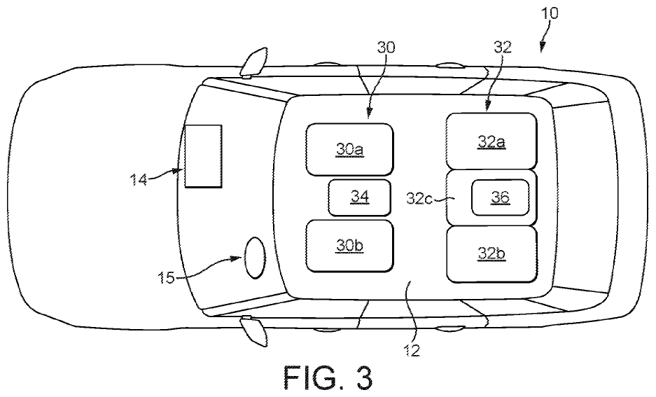

Referring to FIG. 3, inside the vehicle cabin 12 there is provided a first seat arrangement, referred to generally as 30, and a second seat arrangement, referred to generally as 32. The first seat arrangement 30 takes the form of a first row of two front seats 30a, 30b located in a relatively forward position in the vehicle. The first row of front seats includes a passenger's seat 30a on the right hand side of the vehicle and a driver's front seat 30b on the left hand side of the vehicle. In other configurations the driver's seat may be on the right hand side of the vehicle as opposed to the left. A centre console 34 is arranged between the left and right side front seats 30a, 30b of the first row. Conveniently the centre console 34 has a flat upper surface suitable for providing a rest surface for an occupant's arm. The centre console 34 may also provide storage space beneath a lid of the console. The centre console 34 also houses features of a vehicle ventilation system, including left and right side air vents (not shown in FIG. 3), which are located in a rear-facing surface of the centre console 34 to direct air towards, and impact the environment of, occupants seated behind the first row of seats 30.

Rearward of the first row of seats 30, the second seat arrangement 32 takes the form of a second row of three rear seats 32a, 32b, 32c; a rear right seat 32a, a rear left seat 32b and a centre seat 32c. The rear left seat 32b is generally located rearward of the front left seat 30b and the rear right seat 32a is generally located rearward of the front right seat 30a. The centre rear seat 32c is located generally rearward of the front centre console 34. In a known manner, a rear drop-down platform 36 is located over the centre rear seat 32c when the platform is pivotally dropped down from an upright position in which the platform is stowed in a recess (not shown in FIG. 3) in the rear seat arrangement 32.

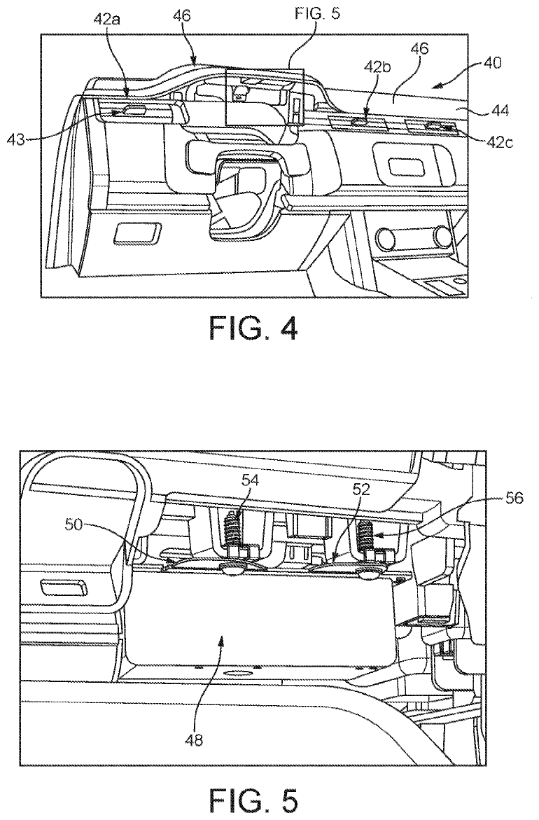

In front of the front row of seats 30 is a vehicle dashboard which includes an instrument panel (not visible in FIG. 3) housing various displays and controls which are accessible to occupants of the vehicle to control and observe various vehicle functions. Referring to FIGS. 4 and 5, the instrument panel 40 is shown in more detail. For example, the instrument panel 40 may house an audio system control, a navigation system control and/or a heating system control. The control system for any or all of the system controlled via the instrument panel is represented as item 14 in FIG. 3 as being behind the instrument panel 40, but it will be appreciated that the one or more controllers of the instrument panel may be located anywhere in the vehicle, and not necessarily behind the instrument panel 40. The instrument panel 40 is provided with a plurality of front air vents 42a, 42, 42c. Fresh air from outside the vehicle, and recirculated air from within the vehicle, can be introduced to or recirculated within the vehicle cabin 12 via the front air vents 42a, 42b, 42c (as well as through rear air vents, as described in further detail below). Further air vents may also be provided in regions of the instrument panel 40 which are not visible in FIG. 5.

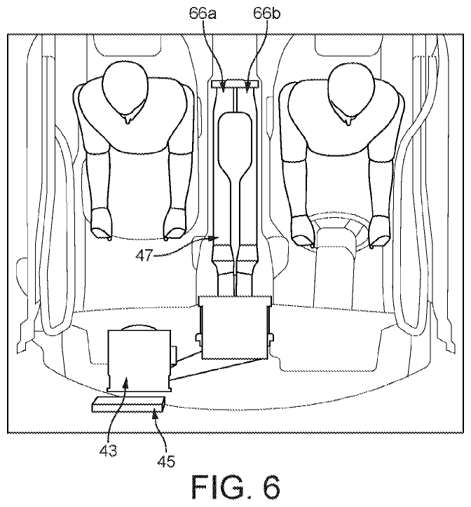

Referring also to FIG. 6, air is delivered into the cabin through the air vents 42a, 42b, 42c via a filtration system including a main filter 43 for filtering particulates from the air and a pre-filter 45 upstream of the main filter 43. The main filtration system 43, 45 communicates with the air vents 42a, 42b, 42c through front air ducting (not shown). The main filter 43 typically includes a particulate filter for filtering particles from the airflow having a size up to 2.5 μm in size. This may be referred to as a “PM2.5” filter. A pre-filter 45 may also be provided upstream of the main filter. The pre-filter 45 may filter particles having a size up to 10 μm in size. This may be referred to as a “PM10” filter. The PM2.5 filter 43 may be provided with an anti-allergen coating technology which is proven to cause viruses and allergens to deactivate, providing further advantages for virus control within the vehicle cabin 12. One or more PM2.5 filters may be provided within the vehicle. The PM2.5 filter(s) may also be provided with an active carbon layer for substantially reducing odour and other harmful gases. The filtration system 43, 45 supplies a filtered air flow via front air ducting through the air vents 42a, 42b, 42c in the instrument panel 40 and also through rear air ducting 47 in the centre console 34 to be expelled through air vents 66a, 66b (described in further detail below) provided in the centre console 34. The filtration system is operable in various modes including a fresh air mode and a recirculation mode, with the mode being selected in dependence on the outputs from various sensors associated with the vehicle, as described in further detail below.

The air flow from the front vents 42a, 42b, 42C is mainly concentrated in the front row seats 30 (this zone is referred to as the front occupant space). Some of this air is also carried over to the second row seats 32, at a lower velocity. Three front air vents 42a, 42b, 42c are shown in FIG. 4, two of which 42a, 42b are associated with the left hand front seat 30b and one of which 42c is associated with the right hand front seat 30a. A fourth air vent (not shown) is also provided for the right hand front seat 30b. Adjustable fins (for example 43) of the air vents 42a, 42b, 42c allow the direction of the airflow into the front occupant space to be adjusted.

The instrument panel 40 includes a top cover 44 including a raised portion 46 which is located forward of the front right seat 30a. A first air purifier device 48 in the form of an OH (hydroxyl) radical generator device (also referred to as an ionisation device) is mounted in a first position within the vehicle to impact or influence air quality in a first region of the vehicle cabin 12 associated with the first seat arrangement 30. The first ionisation device 48 in the present embodiment is mounted beneath the raised portion 46 via mounting portions 50, 52 which are attached to an internal frame of the instrument panel 40 by means of screws 54, 56. The first ionisation device 48 uses the Nanoe™ X technology which uses a high voltage to create trillions of Hydroxyl (OH) radicals enveloped in nano sized water molecules. These OH radicals deactivate pathogens by breaking down virus and bacteria proteins which helps to inhibit their growth. As well as combating pathogens, the OH radicals also act upon odour molecules and allergens in a similar way.

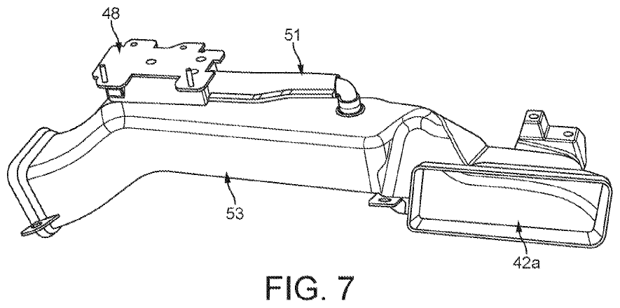

Referring also to FIG. 7, the first ionisation device 48 includes an air inlet (not shown) and an air outlet (also not shown) which communicates with an outlet tube 51 of the first ionisation device 48. The first ionisation device 48 is configured to draw air in through the inlet from the surroundings and then emits OH radicals through the outlet into the outlet tube 51 which communicates with the front air ducting 53. As the front air ducting 53 is arranged to receive the filtered flow of air through the main filtration system 43, 45, this means that the OH radicals are mixed with the filtered air flow in the ducting 53 and together the filtered air flow and the OH radicals are delivered through the air vents 42a,42b,42c (one of which 42a is identified in FIG. 7) into the front occupant space. The first ionisation device 48 therefore impacts mainly the air quality in a first zone or region of the vehicle, being the front occupant space. The first zone or region of the vehicle cabin 12 is associated with the first seat arrangement 30.

The adjustable fins 43 of the air vents 40 then allow the direction of the airflow into the front occupant space to be adjusted.

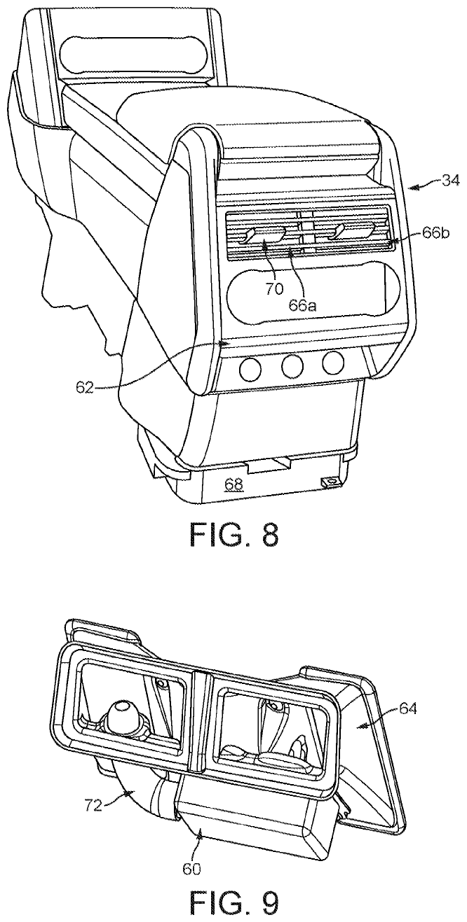

Referring to FIGS. 8 to 10, a second air purifier device 60 in the form of a second OH (hydroxyl) radical generator device (also referred to as an ionisation device) is mounted in a second position within the vehicle to impact or influence air quality in a second region of the vehicle cabin 12 associated with the second seat arrangement 32. The second ionisation device 60 in the present embodiment is mounted in the centre console 34 which is located between the right and left front seats 30a, 30b of the first row of seats. The second ionisation device 60 may take the same form as the first ionisation device which uses the Nanoe™ X technology.

The centre console 34 includes a rear-facing panel 62 which is provided with an opening for receiving an air vent mounting 64. The air vent mounting 64 houses left and right-side adjustable vents 66a, 66b respectively (as seen in FIG. 8), through which a filtered airflow, filtered by the vehicle's filtration system, is delivered to or recirculated within the cabin 12. The air vents 66a, 66b direct air flow towards the cabin space or zone immediately in front of, and in the volume surrounding, occupants of the rear row of seats 32 (referred to as the rear occupant space). Adjustable fins 70 of the air vents 66a, 66b allow the direction of the airflow into the rear occupant space to be adjusted by the occupant. The second ionisation device 60 therefore impacts the air quality in a second zone or region of the vehicle, being the rear occupant space. The second zone or region of the vehicle cabin 12 is associated with the second seat arrangement 32.

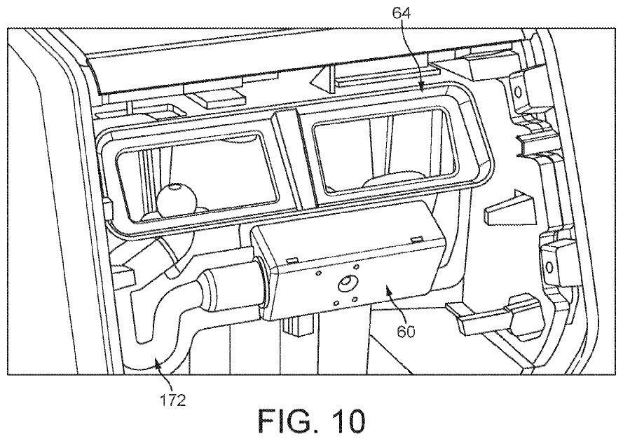

The second ionisation device 60 is mounted behind the air vents 66a, 66b within the centre console 34. The second ionisation device includes an output tube 72 through which the ionised air flow is dispelled from the device 60 into the rear air ducting 47 where it combines with the filtered air flow. The output tube 70 projects up through an opening in the air vent frame 64 to deliver the filtered, ionised air flow out through the left side air vent 66a. The second ionisation device 60 may be mounted in the centre console 34 together with padding/damping material (not shown) which serves to damp noise, vibration and harshness (NVH) effects. In another arrangement, as shown in FIG. 10, the output tube 172 has a more convoluted construction before its exit point through the opening in the air vent frame 64. The arrangement in FIG. 10 has been observed to benefit NVH. Padding/damping material may also be accommodated within the arrangement of FIG. 9.

The filtration system is operable in at least two functions. In a “recirculation” mode of operation (also referred to as the “Purify” mode of operation) the air in the vehicle does not pass through the pre-filter 45 (PM10) but instead will pass many times (being recirculated) through the main filter 43 (PM2.5), whilst being dispelled into the cabin 12 via the various vents, giving a quicker and better filtration efficiency. In a fresh air mode of operation (where the recirculation function is not active), the air enters the vehicle from outside the vehicle and passes through the pre filter (PM10) and the main filter (PM2.5) once before entering the cabin via the various vents. Details of the different functions for the filtration system are described in further detail below.

The air purification system also includes a carbon dioxide management device (not shown) which is configured to manage the levels of carbon dioxide within the vehicle cabin dependent on control by the user and/or optionally based on feedback from a carbon dioxide sensor.

The controller 14 for the air purification system is arranged to receive a combination of sensor signals which are derived from a plurality of vehicle sensors and also user commands, in response to which the air purification system is controlled. The sensor signals are typically derived from sensors mounted internally and externally to the vehicle cabin to measure various air quality parameters (AQPs) inside and outside the vehicle.

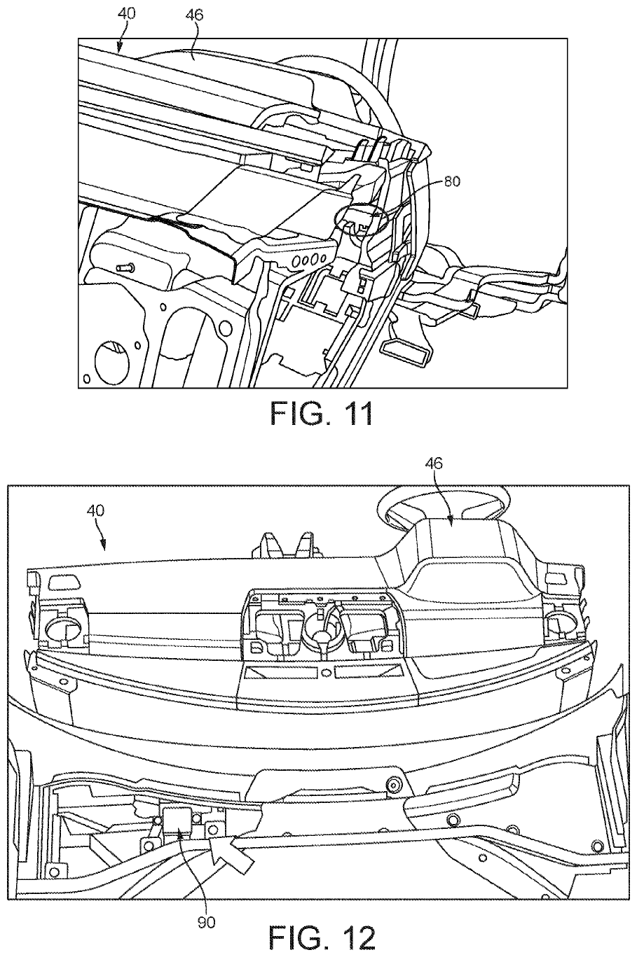

Referring to FIG. 11, the vehicle is typically fitted with a combined particle and carbon dioxide sensor 80 which is mounted inside the vehicle, behind the instrument panel 40. The combined particle and carbon dioxide sensor 80 is arranged to detect the concentration level of particles in the cabin (with a size up to 2.5 μm) that can cause illness, irritation, allergic reaction, and which can also harbour viruses. The sensor 80 also measures the concentration level of carbon dioxide in the cabin. Exposure to carbon dioxide can produce a variety of adverse health effects, including headaches, dizziness and drowsiness and so it is beneficial to measure this and operate the air purification system in response, to reduce the risk of these effects Typically, the sensor 80 may be referred to as a “PM2.5 sensor”. The output from the combined particle and carbon dioxide sensor 80 is provided as a first input to the air purification system controller 14.

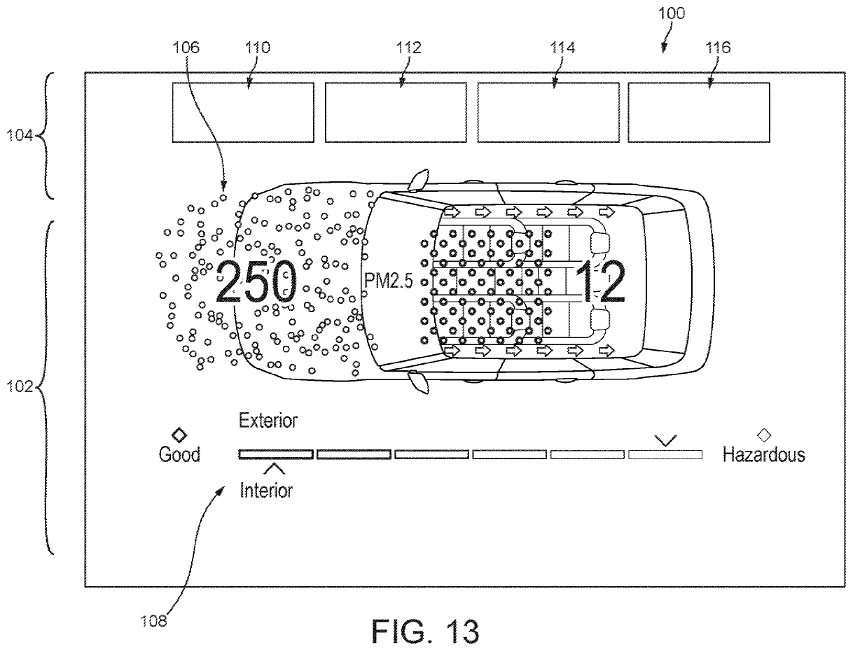

Referring to FIG. 12, the vehicle may also be fitted with particle sensor 90 mounted externally to the vehicle cabin 12. The sensor 90 is fitted on the left hand side of the vehicle (looking from the outside into the vehicle cabin 12) and typically measures the concentration level of particles in the air flow as it enters the vehicle having a diameter of up to 2.5 μm. Typically, the sensor 90 may be referred to as a “PM2.5 sensor”. The output from the particle sensor 90 outside the vehicle is provided as a second input to the air purification system controller 14.

A further air quality sensor (not shown) may also be provided to detect temporary increases in harmful gases.

The air purification system controller 14 receives the signals from the various sensors 80, 90 (and any further sensors) and, in response to the sensor signals, controls features of the air purification system relating to the recirculation function. For example, the main filter 43 and the pre-filter 45 are controlled in response to the sensor signals so as to switch between the recirculation mode and the fresh air mode, automatically selecting the most appropriate mode.

The air purification system is also operable by means of the occupants or users of the vehicle in the following manner, so as to control whether the first and second ionisation devices 48, 60 are activated.

Referring to FIG. 13, the air purification system also includes a user input arrangement in the form of a touch-sensitive input device 100, which is configured to receive user commands from an occupant or user of the vehicle. The instrument panel 40 at the front of the vehicle 10 accommodates the input device 100 which includes a display region 102 and a user input region 104. The display region 102 displays a plan view 106 of the vehicle and provides an indication to the vehicle occupants of the air quality distribution throughout the vehicle cabin 12, based on measurements relating to the outputs from the various sensors (e.g. sensors 80, 90). The display region 102 also includes a sliding scale 108 which represents the air quality distribution in the plan view 106. The scale reflects the health effect of the levels detected by the PM2.5 sensor.

The user input region 104 includes a plurality of user input elements 110, 112, 114, 116, in the form of touch-sensitive regions for receiving a user command to control various functions of the air purification system. By way of example, the first input element 110 is configured to turn on the sensors 80, 90 and, hence, activates the main filter 43 and the carbon dioxide management device in either the recirculation or the fresh air mode, based on the measured levels of particulates and carbon dioxide detected by the sensors. Selection of the first input element 110 also activates the first and second ionisation devices 48, 60. When the first input element 110 is activated by the user, the first and second ionisation devices 48, 60 are therefore turned on together with the carbon dioxide management device and the main filter 43 (and optionally the pre-filter 45), with the mode of operation for the filter(s) 43, 45 being dependent on the sensor measurements.

When the first user input element 110 is selected and the particulate sensor 80 inside the cabin is activated, if the particulate levels inside the cabin 12 are detected at a level greater than a predetermined threshold level, the recirculation mode is activated so that the air passes through the main filter 43 many times. If, on the other hand, the particulate sensor 80 inside the cabin 12 indicates that the particulate levels inside the cabin are below the predetermined threshold level, the fresh air mode is activated so that air is drawn in through the pre-filter 45 and the main filter 43 only once before entering the cabin through the vents. This is the “Purify” function of the system, as mentioned previously, where all four devices may be activated simultaneously (first and second ionisation devices 48, 60, carbon dioxide management device and main filter 43).

The second input element 112 is configured to control the first and second ionisation devices 48, 60, so that when it is activated only the first and second ionisation devices 48, 60 are activated but not the carbon dioxide management device. This may be referred to as the “Ionise” function of the system, which does not turn on the Purify function (whether recirculation mode or fresh air mode).

The third input element 114 is configured to control only the carbon dioxide management device so that when it is activated only the carbon dioxide management device is activated. The fourth input element 116 is operated by the user when it is desired to access air quality data (e.g. pollutants) received over a wireless network (e.g. cloud-based data), for example relating to the air quality in the area surrounding the vehicle or in the area at a destination for the vehicle.

The input device 100, being located in the front of the vehicle, is referred to as the front user input arrangement and is located to allow convenient operation by users of the vehicle occupying the first row of seats. The input device 100 is typically a wired device which connects to the controller 14 through electrical wiring. In other embodiments, the front user input arrangement may be a Bluetooth® device which connects the controller via a wireless connection.

It is a particular feature of the present invention that the second ionisation device 60 is located in the centre console 34 and is configured to direct a purified air flow into the zone associated with occupants of the vehicle in the rear row of seats 32, impacting the rear occupant space. As described previously, operation of the second ionisation device 60 in combination with the first ionisation device 48 may be operated by an occupant in the front row of seats operating either the first or second user input element 110, 112. Furthermore, because the second ionisation device 60 is accommodated within the front centre console 34, and is configured to direct a clean airflow into the rear occupant space, the air quality distribution within the vehicle cabin is of a much more homogeneous nature compared to vehicles having only one ionisation device behind the front instrument panel.

In addition to the front user input device 100 for occupants of the vehicle in the front seats 30, the occupants in the rear row of seats 32 are provided with a rear user input arrangement or device 120 which can be used to control functions of the air purification system in the same way as the front user input device 100. The operation of the first ionisation device 48 and the second ionisation device 60 can be selectively controlled in dependence on operation of the front user input device 100 and/or the rear user input device 120. The front user input device 100 and the rear user input device 120 are configured to receive user commands to control operation of the first ionisation device 48 and the second ionisation device 60. Operation of the second ionisation device 60 in combination with the first ionisation device 48 may be activated by means of the user elements of the second user input device 120. This provides maximum convenience of use for the vehicle users and avoids the need for the driver of the vehicle to be the only one controlling the air purification functions.

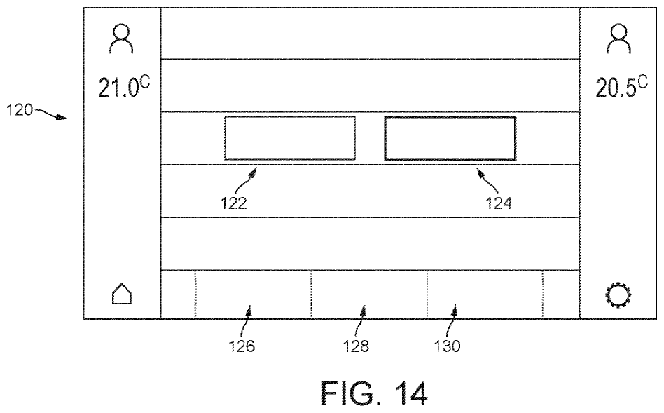

In more detail, and referring to FIG. 14, the user input device 120 for the rear row of seats 32 includes first and second user input elements, 122, 124 respectively, in the form of first and second touch-sensitive regions. The first user input element 122 of the rear user input device 120 is configured to control the carbon dioxide management device and the filtration system mode (recirculation or fresh air), as well as the first and second ionisation devices 48, 60. When the first user input element 122 is activated by the user, the first and second ionisation devices 48, 60 are therefore turned on together (and together with features of the filtration system, depending on the sensor measurements). This is the “Purify” function of the system described previously. The first input element 122 of the rear user input device 120 therefore has the same functionality as the first input element 110 of the front user input device 100.

The second user input element 124 of the rear user input device 120 is configured to control the first and second ionisation devices 48, 60, so that when it is activated only the first and second ionisation devices 48, 60 are activated, but the sensors are not activated and so the Purify function is not turned on. The second user input element 124 of the rear user input device 120 therefore has the same functionality as the second input element 112 of the front user input device 100.

The provision of the rear user input device 120 provides convenience for the vehicle users and means that the responsibility for operation of the air purification system can be passed to occupants in the rear of the vehicle whilst the driver may be otherwise engaged.

Conveniently, and as illustrated in FIG. 14, the rear user input device 120 may take the form of a mobile input device, such as a personal tablet, which communicates with the controller 14 via a wireless (e.g. Bluetooth®) connection. The personal tablet may be mountable within a suitable mount provided on the rear centre console (36—as in FIG. 3) or may be mountable within the rear-facing surface of one of the seats of the first row 30. In other embodiments, the rear user input device 120 may be a permanent feature in the rear of the vehicle cabin 12, located in a convenient position for operation by the rear-seated occupants. In other embodiments, the rear user input device 120 may be a wired device which is located within the vehicle permanently. Whatever the connectivity function, the location of the rear input device 120 is selected to be accessible conveniently by a user of the vehicle so that the controls of the rear input device 120 are suitable for use by an occupant in the rear of the vehicle.

As well as the first and second user input elements 122, 124, the mobile input device includes first, second and third controls 126, 128, 130 for selecting to display information about, or control, features relating to the climate within the vehicle, the vents within the vehicle and the air quality within the vehicle, respectively. In the illustration shown the air quality function is selected to display the first and second user input elements 122, 124 for the “Purify” function and the “Ionisation” mode of the system.

In other embodiments, the air purification system may be operable remotely from the vehicle via a user input device (such as the mobile tablet 120) which may be carried by a vehicle user. For example, in advance of getting into the vehicle, the user may operate a user input device 120 to activate whichever features of the air purification system are required, before entering the vehicle. The system may be activated on approach to the vehicle, or alternatively may be activated when the user is at home and some time prior to entering the vehicle, allowing the vehicle cabin to be fully prepared with a clean and homogenous air quality environment before entry.

It will be appreciated that if the user input device for the rear row of seats is a mobile device, it may also be operated by a user in the front row of seats as the device is portable.

In other embodiments, prior to leaving the vehicle the user may activate an air purification function such that once the car is locked and empty of users, an air quality deep cleaning cycle (removal of virus, harmful gases etc), may be initiated to cleanse the cabin environment prior to next use.

In addition to the ionisation and filtration systems within the vehicle, the sensors of the system may also be used to automate control of the system to benefit the air quality of the environment within the vehicle cabin. For example, feedback signals from the carbon dioxide sensor and the air quality sensor may be used for automated control purposes. Occupancy sensing may also be provided as an input to the purification system so that the system is operable in response to the occupancy status/location within the vehicle.

It will be appreciated that various other embodiments of the air purification system are envisaged without departing from the scope of the accompanying claims.

1. An air purification system for a vehicle cabin of a vehicle comprising a first seat arrangement and a second seat arrangement, the air purification system comprising:
a first ionisation device mountable in a first position within the vehicle to impact air quality in a first region of the vehicle cabin associated with the first seat arrangement;
a second ionisation device mountable in a second position within the vehicle to impact air quality in a second region of the vehicle cabin associated with the second seat arrangement;
a user input arrangement configured to receive at least a first user command; and
a control system comprising one or more controllers, the control system configured to receive the first user command and generate a first control signal in response to the first user command to operate the air purification system in a first mode of operation in which both the first ionisation device and the second ionisation device are activated together.

2. The air purification system of claim 1, wherein the one or more controllers collectively comprise:
at least one electronic processor having an electrical input for receiving the first user command; and
at least one memory device electrically coupled to the at least one electronic processor and having instructions stored therein;
wherein the at least one electronic processor is configured to access the at least one memory device and execute the instructions thereon so as to generate a first control signal in response to the first user command to operate the air purification system in a first mode of operation in which both the first ionisation device and the second ionisation device are activated together.

3. The air purification system as claimed in claim 1, further comprising an air filtration system having a first filter and a second filter, the air filtration system being operable in at least one of a recirculation mode in which air drawn into the vehicle cabin is recirculated through the second filter at least once and a fresh air mode in which air drawn into the vehicle cabin passes through the second filter only once, and a sensor system which generates a sensor output at least in response to at least one of a concentration level of particulates within the vehicle cabin and a concentration of particulates within the external environment, the controller being configured to operate the air filtration system automatically in either the recirculation mode or the fresh air mode in response to the sensor output and the first user command.

4. The air purification system as claimed in claim 3, wherein the user input arrangement is configured to receive a second user command, the controller being configured to receive the second user command and generate a second control signal in response to the second user command to operate the air purification system in a second mode of operation in which the first ionisation device and the second ionisation device are operated together with the air filtration system.

5. The air purification system as claimed in claim 3, wherein the filtration system includes a main filter for filtering particles having a size of up to 2.5 μm.

6. The air purification system as claimed in claim 3, comprising at least one carbon dioxide management device located in at least one of the first and second positions.

7. The air purification system as claim in claim 6, wherein the controller is configured to generate a second signal in response to the second user command to operate the air purification system in a second mode of operation in which the first ionisation device and the second ionisation device are operated together with the air filtration system and the carbon dioxide management device.

8. The air purification system as claimed in claim 7, wherein the user input arrangement includes at least first and second controls for receiving respective ones of the first and second user commands to operate the air purification system in the first and the second mode of operation, respectively.

9. The air purification system as claimed in claim 6, wherein the user input arrangement is configured to receive a third user command, the controller being configured to receive the third user command and generate a third control signal in response to the third user command to operate the air purification system in a third mode of operation in which carbon dioxide management device is operated on its own.

10. The air purification system as claimed in claim 9, wherein the user input arrangement includes a third control for receiving the third user command to operate the air purification system in the third mode of operation.

11. A vehicle comprising:
a vehicle cabin;
a first seat arrangement;
a second seat arrangement; and
an air purification system comprising:
a first ionisation device mountable in a first position within the vehicle to impact air quality in a first region of the vehicle cabin associated with the first seat arrangement,
a second ionisation device mountable in a second position within the vehicle to impact air quality in a second region of the vehicle cabin associated with the second seat arrangement, and
a user input arrangement configured to receive at least a first user command, and
a control system comprising one or more controllers, the control system configured to receive the first user command and generate a first control signal in response to the first user command to operate the air purification system in a first mode of operation in which both the first ionisation device and the second ionisation device are activated together.

12. The vehicle as claimed in claim 11, wherein the first ionisation device is located behind an instrument panel forward of the first row of vehicle seats.

13. The vehicle as claimed in claim 12, wherein the second ionisation device is located in a centre console between adjacent seats of the first row of vehicle seats.

14. The vehicle as claimed in claim 11, wherein the user input arrangement includes a first user input device operable by a user seated in one of the seats of the first row and a second user input device operable by a user seated in one of the seats of the second row, and wherein both the first and second user input elements are configured to receive a first user command to operate the air purification system in the first mode of operation in which both the first ionisation device and the second ionisation device are operated together.

15. A method of controlling an air purification system for a vehicle cabin of a vehicle comprising a first seat arrangement and a second seat arrangement, a first ionisation device mountable in a first position within the vehicle to impact air quality in a first region of the vehicle cabin associated with the first seat arrangement and a second ionisation device mountable in a second position within the vehicle to impact air quality in a second region of the vehicle cabin associated with the second seat arrangement; the method comprising receiving, at a controller, a first user command which is operable to control the first ionisation device and the second ionisation device in a first mode of operation in which they are both activated together.

16. The vehicle as claimed in claim 11, wherein the first seat arrangement is a first row of vehicle seats and the second seat arrangement is a second row of vehicle seats behind the first row of vehicle seats.

17. The vehicle as claimed in claim 12, wherein the second ionisation device is located in a region of the vehicle cabin between adjacent seats of the first row of vehicle seats.

18. The vehicle as claimed in claim 13, wherein the second ionisation device is located behind an air vent located in the centre console.
